(19) United States
(12) Patent Application Publication
(10) Pub. No.: US 2025/0321173 A1
(43) Pub. Date: Oct. 16, 2025

(54) A TEST DEVICE FOR MEASUING LIQUID ABSORPTION CAPACITY

(71) Applicant: ACIBADEM MEHMET ALI AYDINLAR UNIVERSITESI, Istanbul (TR)

(72) Inventors: Kutalp KURT, Istanbul (TR); Erdi Gorkem DIRILEN, Istanbul (TR); Ozgul GOK, Istanbul (TR); Mehmet Ilhan GOLHAN, Istanbul (TR); Ceyhun Ekrem KIRIMLI, Istanbul (TR)

(73) Assignee: ACIBADEM MEHMET ALI AYDINLAR UNIVERSITESI, Istanbul (TR)

(21) Appl. No.: 18/869,985

(22) PCT Filed: Jun. 2, 2023
(86) PCT No.: PCT/TR2023/050513

§ 371 Date: Nov. 27, 2024

(30) Foreign Application Priority Data
Jun. 3, 2022 (TR) 2022/009138

(51) Int. Cl. G01N 5/02; G01N 35/00

(52) U.S. Cl. CPC G01N 5/02; G01N 35/00871; G01N 2035/00891

(57) ABSTRACT

The present invention relates to a test device for measuring liquid absorption (swelling) capacity of polymers on a mass basis automatically.

### TECHNICAL FIELD

The present invention relates to a test device for measuring liquid absorption (swelling) capacity of polymers on a mass basis automatically.

### BACKGROUND OF THE INVENTION

The hydrogel sector is a rapidly growing sector, particularly for the last 10 years, with a worldwide value of billions of dollars. Hydrogels are polymers exhibiting high level of swelling property and thus their use in the biomedical and cosmetic sectors are quite high. A procedure called “Swelling Test (ST)” is currently applied to measure the swelling property which is the most important property of hydrogels. In this procedure, mass of a dry hydrogel is obtained at first and then this gel is deposited into a liquid at certain time intervals and the mass of the gel is obtained following each interval. When the mass of the gel starts remaining unchanged, the test is terminated and a mass/time graph is obtained. The main problem of this test is that it takes too much time of scientists. Termination of the swelling test of some gels may take hours and this means standing over a balance for hours for a scientist who wants to take a measurement every minute. In addition, collecting the water accumulated on the surface of the gel between each measurement and obtaining mass data mean additional time as well. Presence of high manual error is the second problem of this test. The fact that deformation is found in a gel that is removed from a beaker by means of a scraper or division of gel are quite common conditions and this may invalidate a swelling test. It is also quite possible for a scientist who practices this test repetitively for long periods of time to make a mistake in measurements of mass and time. The fact that a test cannot be performed for long periods of time is the last problem of this test. A person cannot be expected to measure a gel that reaches its maximum swelling capacity in 24 hours, every 5 minutes.

Currently, there is no device which can automatically perform mass-based measurement of hydrogel and other non-superabsorbent materials with swelling property in the sector.

Therefore, there is need for a mass-based test device which aims to get rid of the current defects of a swelling test and which will not take hours of scientists and will eliminate high human error in a swelling test and also can automatically perform the tests that are impossible for humans to perform.

The International patent document no. WO0239093, an application included in the state of the art, discloses measuring a tube piece-which rises by applying a compressive force to a superabsorbent gel and then the gel resists the compressive force based on swelling-by means of CCD optics. A volumetric measurement is taken based on this change of height as well. However, immersing a superabsorbent gel in water by means of mechanical methods and taking mass-based measurements by using a balance based on the liquid mass varying in the liquid container are not disclosed.

In the Chinese patent document no. CN102313756, an application in the state of the art, swelling kinetics of coal is measured. However, this mechanism measures the thermal swelling of a material by means of the height of a plate rising based on volume. Polymers with swelling property swell by volume as they absorb water and they yield less precise result in comparison to direct mass measurements due to the fact that the mass change to be calculated on the basis of this volumetric difference ignores the pore structure of the polymer, i.e. the information on how much space is included in the pore, in other words it is an indirect measurement. Therefore, a device which provides access to the information of precise liquid absorption capacity by means of direct measurements—that use mass as a base instead of indirect measurement—is not mentioned.

### SUMMARY OF THE INVENTION

An objective of the present invention is to realize a test device which measures liquid absorption capacity of polymers on a mass basis automatically.

Another objective of the present invention is to realize a test device which does not take too much time of scientists, eliminates errors of measurement that are carried out manually, and enables to automatically perform tests that require long periods.

Another objective of the present invention is to realize a test device which provides more precise measurement results in comparison to data of liquid absorption capacity that are based on volume-based measurements.

Another objective of the present invention is to realize a test device which detects the liquid absorption capacity directly by means of mass data, not by volume indirectly.

### DETAILED DESCRIPTION OF THE INVENTION

“Test Device for Measuring Liquid Absorption Capacity” realized to fulfil the objectives of the present invention is shown in the figures attached, in which:

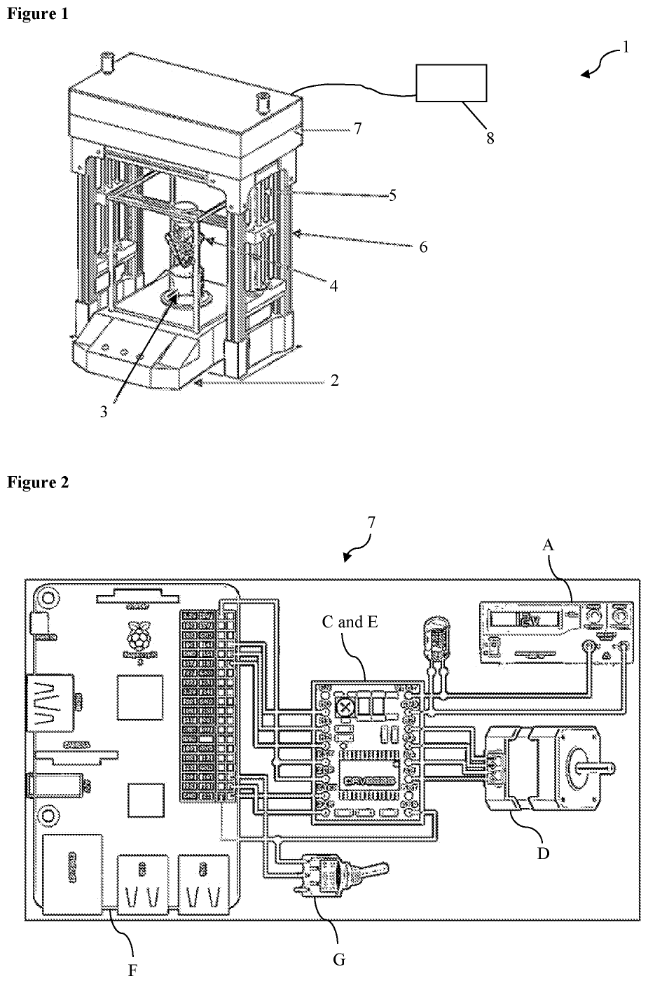

FIG. 1 is a front perspective view of the inventive test device.

FIG. 2 is a view indicating the interconnections of the components included on the cover in the inventive test device.

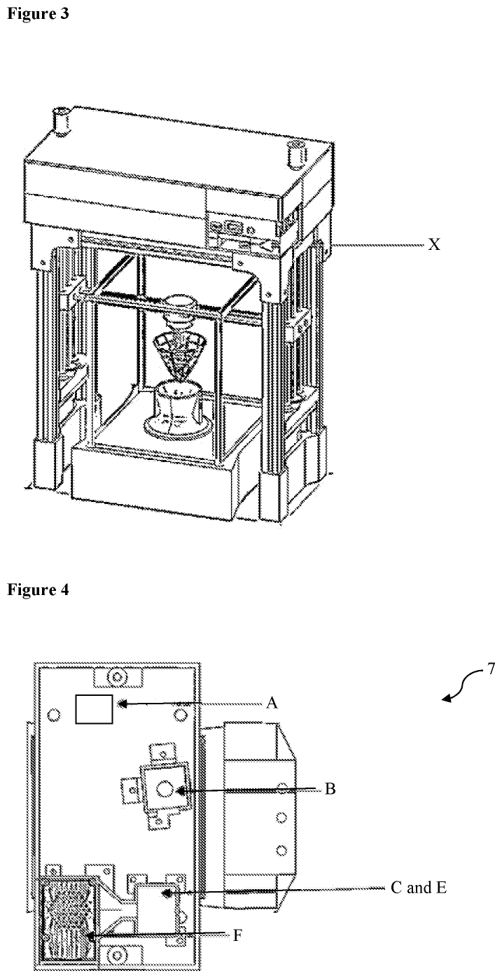

FIG. 3 is a rear perspective view of the inventive test device.

FIG. 4 is a view indicating the components included on the cover in the inventive test device.

The components illustrated in the figure are individually numbered, where the numbers refer to the following:
1. Test device
2. Balance
3. Container
4. Sample housing
5. Sliding member
6. Framework
7. Cover
8. Electronic device
A. 12V power unit
B. Stepper motor housing
C. Stepper motor driver
D. Stepper motor
E. Breadboard
F. Single-board computer
G. Plug
X. USB and HDMI inputs

The inventive test device (1) for measuring liquid absorption capacity of polymers on a mass basis automatically comprises
at least one balance (2) which is used for mass measurement;
at least one container (3) which is placed on the balance (2) and wherein liquid is put;
at least one sample housing (4) wherein the sample to be immersed in the liquid inside the container (3) is placed;
at least one sliding member (5) which allows movement in a vertical direction so as to immerse the sample housing (4) in the container (3) and remove it;
at least one framework (6) which supports the sliding member (5) to perform sliding movement in a vertical direction;
at least one cover (7) which is located on the upper part of the framework (6) and comprises connection inputs, motor, power unit (A) and device's brain; and
at least one electronic device (8) which establishes connection by means of connection inputs in the cover (7) and enables to input a parameter related to a test and to view results of measurements.

The balance (2) included in the inventive test device (1) is configured to measure the mass data of the polymer/gel mass, that is the sample whose liquid absorption capacity is aimed to be measured, before and after it is immersed into liquid. The balance (2) has a surface suitable for placing the container (3) thereon.

The container (3) included in the inventive test device (1) is configured to ensure that the liquid to be absorbed by the sample, that is a gel or polymer and whose liquid absorption capacity is aimed to be determined, is kept. The container (3) is placed on the surface located on the balance (2) for the test procedure. In one embodiment of the invention, the container (3) is a beaker.

The sample housing (4) included in the inventive test device (1) is configured to ensure that the sample whose liquid absorption capacity is aimed to be determined is kept. The sample housing (4) is configured to be able to move on the container (3) in a vertical direction by means of the sliding member (5). The sample housing (4) has dimensions suitable to fit into the container (3) in order to ensure that the sample placed in thereof can contact the liquid inside the container (3).

The sliding member (5) included in the inventive test device (1) is in connection with the sample housing (4) and is configured to keep the sample in a liquid by moving it on the framework (6) upwards and downwards and then remove it and to ensure that the necessary movement is performed so as to repeat this process at a predetermined frequency.

In one embodiment of the invention, the framework (6) included in the inventive test device (1) has a rectangular shape with an open short edge. The framework (6) is placed such that the inner part of its closed short edge faces the container (3) on the balance (2) and the ends of the open edge face on the same ground with the balance (2) (FIG. 1). The framework (6) is configured to enable the sliding member (5) to perform upwards and downwards sliding movement on the long edges of the rectangular shape.

The cover (7) included in the inventive test device (1) is placed on the surface of the closed short-edge rectangular part of the framework (6) that does not face the balance (2). The cover (7) comprises USB and HDMI inputs (X) in order to ensure that connection is established with the electronic device (8). The cover (7) comprises at least one 12V power unit (A), at least one stepper motor housing (B), at least one breadboard (E), at least one stepper motor driver (C), at least one stepper motor (D) and at least one single-board computer ((device's brain) Raspberry Pi) (F) on thereof. The cover (7) is configured to enable the electronic device (8) to establish connection with the keyboard and the mouse by means of the USB input (X) located on thereof. The cover (7) is configured to enable the balance (2) to connect to the serial port by means of the USB input (X) located on thereof. The cover (7) is configured to enable the electronic device (8) to establish connection with the screen by means of the HDMI input (X) located on thereof. The cover (7) is configured to ensure that the 12V power unit (A) and the Raspberry Pi (F) located on thereof are connected to a plug (G). The cover (7) is configured to ensure that the stepper motor driver (C), the breadboard (E), the 12V power unit (A), the stepper motor (D) and the Raspberry Pi (F) located on thereof are interconnected. A test is initiated automatically in accordance with the predetermined parameters by means of the Raspberry Pi (F) located on the cover (7) and the test is terminated according to the predetermined parameters. The amount of mass measured in the balance (2) is kept under record by means of the Raspberry Pi (F) located on the cover (7) and results are obtained in relation to the liquid absorption capacity.

The electronic device (8) included in the inventive test device (1) comprises at least one keyboard, at least one mouse and at least one screen. The electronic device (8) is configured to enter data such as how many times the sample, that is placed in the sample housing (4) in order to measure the liquid absorption capacity by means of the keyboard, will be immersed in the container (3) or the lower limit of mass change, the immersion depth, the swelling time and the mass of the sample determined for terminating the test. The electronic device (8) is configured to ensure that the data entered over the keyboard are transmitted to the Raspberry Pi (F). The electronic device (8) is configured to ensure that command entry is made for performing the determination test of liquid absorption capacity automatically. The electronic device (8) is configured to ensure that the result data about to the determination test of liquid absorption capacity performed are displayed over the screen.

In one embodiment of the invention, the container (3) is filled with water and balance (2) then placed on the balance (2). The sample housing (4) is wet. The user enters the parameter such as the mass, the number of repetitions, the swelling time and the immersion depth of the sample-that is a dry gel or polymer—to the electronic device (8) by means of the keyboard. Then, the user leaves the sample inside the sample housing (4) and initiates the test. Upon setting the balance (2) to zero, the test device (1) immerses the sample in water as much as the specified immersion depth and removes the sample from the water after keeping it under water until the swelling period. The amount of decreasing sample amount is recorded in the balance (2). This process is repeated as many times as the number of repetitions. When the measured mass change drops below a level specified by the user, the test is terminated automatically. The user receives the sample and swelling test data from the test device (1) when the test is finished.

With the inventive test device (1), a mass-based automatic test can be performed for the samples whose liquid absorption (swelling) capacity takes much time and also which takes too much time of scientists. The swelling test results, which are obtained by high manual error are replaced by more accurate and precise results.

Within these basic concepts; it is possible to develop various embodiments of the inventive test device for measuring liquid absorption capacity (1); the invention cannot be limited to examples disclosed herein and it is essentially according to claims.

1. A test device (1) for measuring liquid absorption capacity of polymers on a mass basis automatically; comprising
at least one balance (2) which is used for mass measurement; characterized by
at least one container (3) which is placed on the balance (2) and wherein liquid is put;
at least one sample housing (4) wherein the sample to be immersed in the liquid inside the container (3) is placed;
at least one sliding member (5) which allows movement in a vertical direction so as to immerse the sample housing (4) in the container (3) and remove it;
at least one framework (6) which supports the sliding member (5) to perform sliding movement in a vertical direction;
at least one cover (7) which is located on the upper part of the framework (6) and comprises connection inputs, motor, power unit (A) and device's brain; and
at least one electronic device (8) which establishes connection by means of connection inputs in the cover (7) and enables to input a parameter related to a test and to view results of measurements.

2. A test device (1) according to claim 1; characterized by the balance (2) which is configured to measure the mass data of the polymer/gel mass, that is the sample whose liquid absorption capacity is aimed to be measured, before and after it is immersed into liquid.

3. A test device (1) according to claim 1 or 2; characterized by the balance (2) which has a surface suitable for placing the container (3) thereon.

4. A test device (1) according to claim 1; characterized by the container (3) which is configured to ensure that the liquid to be absorbed by the sample, that is a gel or polymer and whose liquid absorption capacity is aimed to be determined, is kept.

5. A test device (1) according to claim 1; characterized by the container (3) which is placed on the surface located on the balance (2) for the test procedure.

6. A test device (1) according to claim 4; characterized by the container (3) which is a beaker.

7. A test device (1) according to claim 1; characterized by the sample housing (4) which is configured to ensure that the sample whose liquid absorption capacity is aimed to be determined is kept.

8. A test device (1) according to claim 1; characterized by the sample housing (4) which is configured to be able to move on the container (3) in a vertical direction by means of the sliding member (5).

9. A test device (1) according to claim 1; characterized by the sample housing (4) which has dimensions suitable to fit into the container (3) in order to ensure that the sample placed in thereof can contact the liquid inside the container (3).

10. A test device (1) according to claim 1; characterized by the sliding member (5) which is in connection with the sample housing (4) and is configured to keep the sample in a liquid by moving it on the framework (6) upwards and downwards and then remove it and to ensure that the necessary movement is performed so as to repeat this process at a predetermined frequency.

11. A test device (1) according to claim 1; characterized by the framework (6) which has a rectangular shape with an open short edge.

12. A test device (1) according to claim 1; characterized by the framework (6) which is placed such that the inner part of its closed short edge faces the container (3) on the balance (2) and the ends of the open edge face on the same ground with the balance (2).

13. A test device (1) according to claim 1; characterized by the framework (6) which is configured to enable the sliding member (5) to perform upwards and downwards sliding movement on the long edges of the rectangular shape.

14. A test device (1) according to claim 1; characterized by the cover (7) which is placed on the surface of the closed short-edge rectangular part of the framework (6) that does not face the balance (2).

15. A test device (1) according to claim 1; characterized by the cover (7) which comprises USB and HDMI inputs (X) in order to ensure that connection is established with the electronic device (8).

16. A test device (1) according to any of the preceding claim 1; characterized by the cover (7) which comprises at least one 12V power unit (A), at least one stepper motor housing (B), at least one breadboard (E), at least one stepper motor driver (C), at least one stepper motor (D) and at least one single- board computer (F) on thereof.

17. A test device (1) according to claim 1; characterized by the cover (7) which is configured to enable the electronic device (8) to establish connection with the keyboard and the mouse by means of the USB input (X) located on thereof.

18. A test device (1) according to claim 1; characterized by the cover (7) which is configured to enable the balance (2) to connect to the serial port by means of the USB input (X) located on thereof.

19. A test device (1) according to claim 1; characterized by the cover (7) which is configured to enable the electronic device (8) to establish connection with the screen by means of the HDMI input (X) located on thereof.

20. A test device (1) according to claim 1; characterized by the cover (7) which is configured to ensure that the 12V power unit (A) and the Raspberry Pi (F) located on thereof are connected to a plug (G).

21. A test device (1) according to claim 1; characterized by the cover (7) which is configured to ensure that the stepper motor driver (C), the breadboard (E), the 12V power unit (A), the stepper motor (D) and the Raspberry Pi (F) located on thereof are interconnected.

22. A test device (1) according to claim 1; characterized by the cover (7) which initiates a test automatically in accordance with the predetermined parameters by means of the Raspberry Pi (F) located on thereof and terminates the test according to the predetermined parameters.

23. A test device (1) according to claim 1; characterized by the cover (7) which enables to keep the amount of mass, that is measured in the balance (2), under record by means of the Raspberry Pi (F) located on the cover (7) and to obtain results in relation to the liquid absorption capacity.

24. A test device (1) according to claim 1; characterized by the electronic device (8) which comprises at least one keyboard, at least one mouse and at least one screen.

25. A test device (1) according to claim 1; characterized by the electronic device (8) which is configured to enter data such as how many times the sample, that is placed in the sample housing (4) in order to measure the liquid absorption capacity by means of the keyboard, will be immersed in the container (3) or the lower limit of mass change, the immersion depth, the swelling time and the mass of the sample determined for terminating the test.

26. A test device (1) according to claim 1; characterized by the electronic device (8) which is configured to ensure that the data entered over the keyboard are transmitted to the Raspberry Pi (F).

27. A test device (1) according to claim 1; characterized by the electronic device (8) which is configured to ensure that command entry is made for performing the determination test of liquid absorption capacity automatically.

28. A test device (1) according to claim 1; characterized by the electronic device (8) which is configured to ensure that the result data about to the determination test of liquid absorption capacity performed are displayed over the screen.
